(19) United States
(12) Patent Application Publication
(10) Pub. No.: US 2013/0319441 A1
(43) Pub. Date: Dec. 5, 2013

(54) DURABLE SPARKLING FINGERNAIL COATING AND METHOD OF FABRICATION THEREFOR

(71) Applicant: Linda Ma, Las Vegas, NV (US)

(72) Inventor: Linda Ma, Las Vegas, NV (US)

(21) Appl. No.: 13/486,381

(22) Filed: Jun. 1, 2012

(51) Int. Cl. A45D 29/11; A61K 8/03

(52) U.S. Cl. 132/200; 424/61; 424/401

(57) ABSTRACT

A sparkling fingernail coating constructed in a multilayer process provides a fingernail covering that is aesthetically pleasing and durable. The sparkling fingernail coating may comprise a foundation layer, glitter adhesion layer, and a protective top layer. Each layer may be cured in sequence for one or more predefined periods of time to ensure durability and a predictable lifetime for the sparkling fingernail coating.

### BACKGROUND OF THE INVENTION

1. Field of the Invention

The invention relates to decorative fingernail treatments and applications, and in particular to a durable sparkling fingernail coating and method of application therefor.

2. Related Art

A variety of decorative fingernail treatments are available today. For instance women and men are known to apply nail polish of various colors to their fingernails to improve or create a desired appearance for their fingernails. Nail polish comes in a variety of colors and can be applied by a consumer or by a professional.

In addition to nail polish, more complex fingernail treatments are also available. Typically, these are applied by a professional technician, such as at a nail spa or day spa. These fingernail treatments can range in complexity and cost. A client may schedule periodic nail appointments for such fingernail treatments much like clients do for hair stylists. However, a client seeking a fingernail treatment may not desire the treatment if his or her last treatment is currently in good condition.

From the discussion that follows, it will become apparent that the present invention addresses the deficiencies associated with the prior art while providing numerous additional advantages and benefits not contemplated or possible with prior art constructions.

### SUMMARY OF THE INVENTION

A sparkling fingernail coating is disclosed herein. The sparkling fingernail coating provides an aesthetically pleasing and attractive fingernail coating, while being durable. This durability is achieved via the elements and process of forming the sparkling fingernail coating. The durability reduces the number of fingernail applications a client must undergo and provides predictability with regard to the amount of time the sparkling fingernail coating will “last” before it becomes worn, unsightly, or otherwise undesirable.

As disclosed herein, the sparkling fingernail coating may comprise a variety of configurations. For instance, in one exemplary embodiment, a sparkling fingernail coating for a fingernail may comprise a foundation layer applied to a top surface and tip of the fingernail, a glitter adhesion layer applied to the foundation layer, and a plurality of glitter particles embedded in the glitter adhesion layer. The glitter will typically be embedded while the glitter adhesion layer is partially cured. A transparent top layer may be applied to the glitter adhesion layer. The transparent top layer will typically be applied to the glitter adhesion layer after the glitter adhesion layer is fully cured. It is noted that multiple top layers may be provided such as to increase protection of the glitter adhesion layer, to allow buffing to a smooth finish (by first increasing the thickness of the top layer), or both.

Various combinations of transparent and color tinted layers may be used to form a sparkling fingernail coating. For instance, the foundation layer may be transparent while the glitter adhesion layer is tinted to a particular color. Alternatively, the glitter adhesion layer may also be transparent. It is noted that the layers may vary in thickness as well. For example, the glitter adhesion layer may be a thin or thinner layer relative to the foundation layer (or top layer).

The glitter adhesion layer (and other layers) may be cured by curing device. Also, it is noted that the glitter adhesion layer may be applied to the foundation layer after the foundation layer has fully cured.

In another exemplary embodiment, a sparkling fingernail coating for a fingernail comprises a first layer configured to secure the sparkling fingernail coating to the fingernail, and a second layer configured to carry a plurality of glitter particles therein. Typically the second layer will cover the first layer and is applied to the first layer after the first layer is fully cured.

A plurality of glitter particles may be embedded in the second layer. Typically, the plurality of glitter particles will be embedded after the second layer is partially cured. One or more third layers may cover the second layer and the plurality of glitter particles. Typically, the one or more third layers will be applied to cover the second layer and plurality of glitter particles after the second layer is fully cured.

Various curing devices may be used to cure one or more layers of the sparkling fingernail coating. For example, the second layer may be partially cured and/or fully cured with UV light (or other curing element such as a heating element or fan).

It is noted that the second layer may be tinted to a particular color, while the one or more third layers are transparent. Also, the one or more third layers may be buffed to a smooth finish.

Various methods for applying or manufacturing a sparkling fingernail coating for a fingernail are disclosed herein as well. For instance, one exemplary method for applying a sparkling fingernail coating may comprise applying a foundation layer configured to secure the sparkling fingernail coating to the fingernail to a top surface and tip of the fingernail, covering the foundation layer with a glitter adhesion layer, partially curing the glitter adhesion layer with a curing device for a first period of time to generate a partially cured glitter adhesion layer, and applying a plurality of glitter particles to the partially cured glitter adhesion layer.

The plurality of glitter particles may be embedded into the partially cured glitter adhesion layer. The glitter adhesion layer may then be fully cured with the curing device for a second period of time to generate a fully cured glitter adhesion layer having the plurality of glitter particles embedded therein. The fully cured glitter adhesion layer may then be covered with a transparent top layer configured to protect the glitter adhesion layer.

It is noted that the second period of time for curing will typically be longer than the first period of time. Also, the foundation layer may be fully cured before the glitter adhesion layer is applied. In addition, excess glitter outside a peripheral edge of the fingernail may be removed after the glitter adhesion layer has fully cured. The transparent top layer may be buffed to generate a smooth finish after it has fully cured.

Other systems, methods, features and advantages of the invention will be or will become apparent to one with skill in the art upon examination of the following figures and detailed description. It is intended that all such additional systems, methods, features and advantages be included within this description, be within the scope of the invention, and be protected by the accompanying claims.

### BRIEF DESCRIPTION OF THE DRAWINGS

The components in the figures are not necessarily to scale, emphasis instead being placed upon illustrating the principles of the invention. In the figures, like reference numerals designate corresponding parts throughout the different views.

FIG. 1 is a perspective view of an exemplary sparkling fingernail coating;

FIG. 2A is a cross section view of an exemplary foundation layer;

FIG. 2B is a cross section view of an exemplary glitter adhesion layer;

FIG. 2C is a cross section view of an exemplary glitter adhesion layer having glitter particles applied thereto;

FIG. 2D is a cross section view of an exemplary sparkling fingernail coating; and

FIG. 3 is a flow diagram illustrating an exemplary application process for a sparkling fingernail coating.

### DETAILED DESCRIPTION OF THE PREFERRED EMBODIMENTS

In the following description, numerous specific details are set forth in order to provide a more thorough description of the present invention. It will be apparent, however, to one skilled in the art, that the present invention may be practiced without these specific details. In other instances, well-known features have not been described in detail so as not to obscure the invention.

In general, the sparkling fingernail coating disclosed herein is a durable fingernail coating having a long lasting finish and consistent lifetime. This is advantageous in that it reduces the number of applications a client must undergo as well as associated costs in time and money. In addition, the application process herein results in a predictable lifetime for the coating, allowing clients to schedule or plan for a next fingernail coating application. As its name implies the sparkling fingernail application is also aesthetically pleasing and attractive.

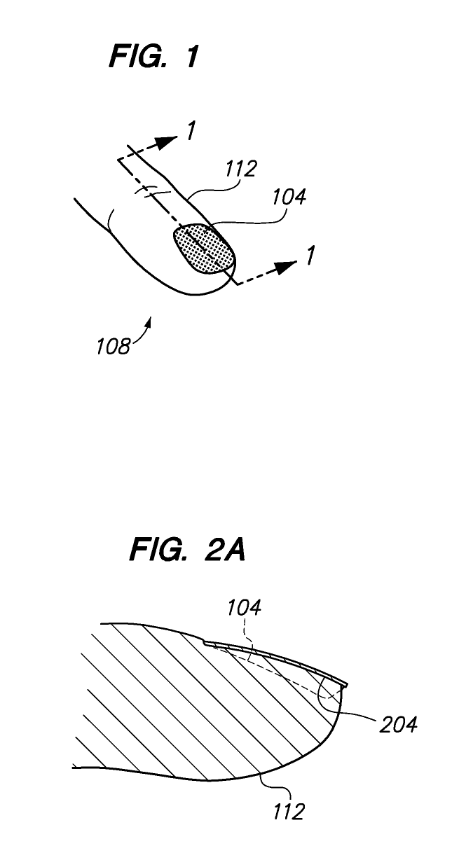

The sparkling fingernail coating will now be described with regard to the drawings of FIGS. 1-3. FIG. 1 illustrates a completed or finished sparkling fingernail coating 108 applied to the fingernail 104 of a client's finger 112. As can be seen and as will be described further below, the sparkling fingernail coating 108 includes a suspension of glitter or other sparking particles applied in a multistep process.

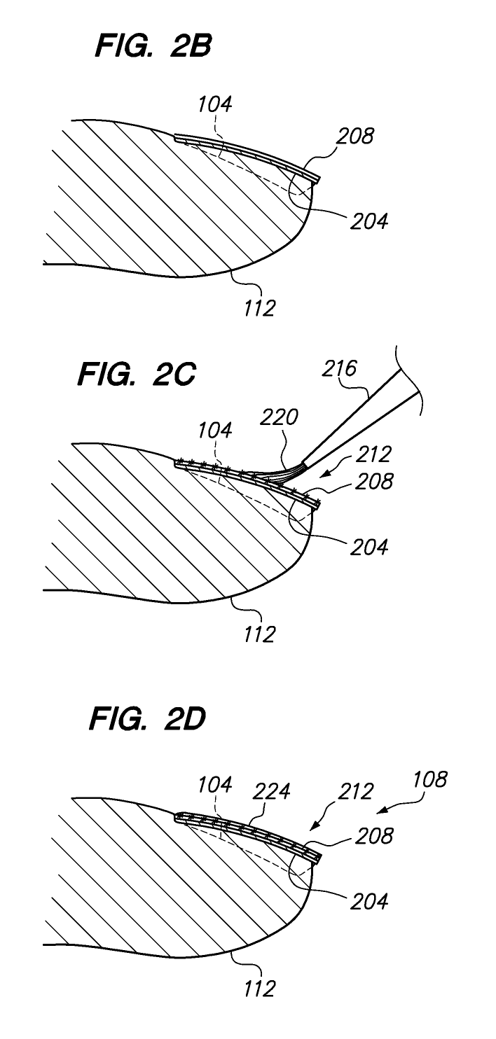

FIGS. 2A-2D illustrate elements of the sparkling fingernail coating 108. In FIGS. 2A-2D, the sparkling fingernail coating 108 is shown via a cross sectional view across Line 1 of FIG. 1. As can be seen and as will be described further below, the sparkling fingernail coating 108 may include one or more layers or sections that, in combination, provide the predictable lifetime and durability along with a eye catching and aesthetically pleasing appearance, as discussed above.

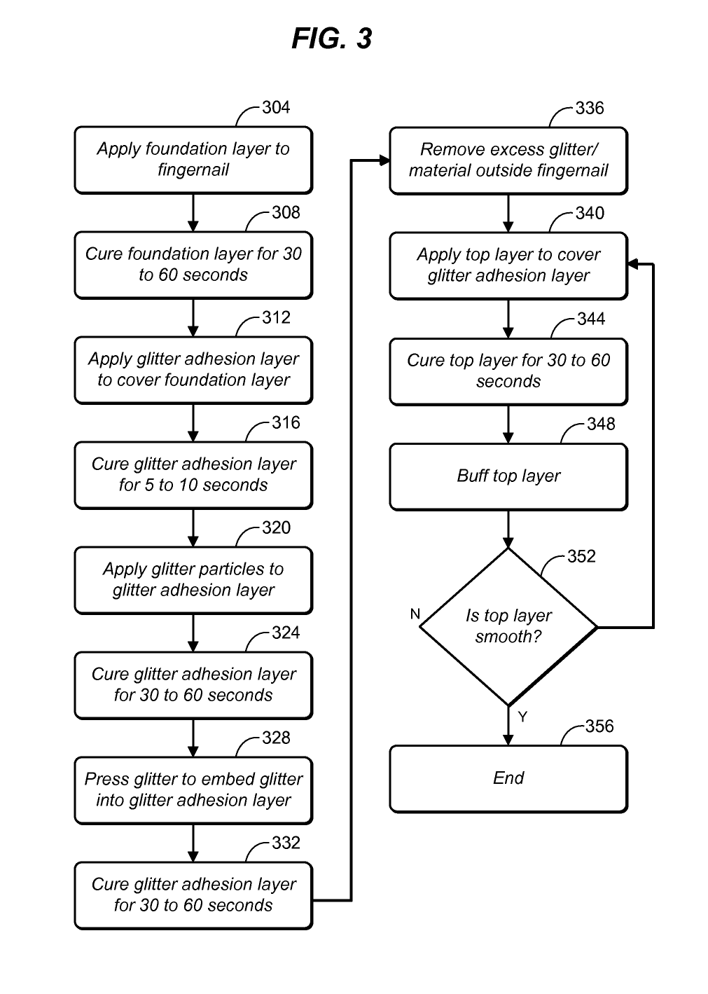

The application process that creates the sparkling fingernail coating 108 will now be described with regard to the flow diagram of FIG. 3, which illustrates an exemplary application process for the sparkling fingernail coating. In the following, the application process is described with reference to FIGS. 2A-2D.

At a step 304, a foundation layer 204 may be applied to a fingernail 104, such as shown in FIG. 2A. The foundation layer 204 will typically be a liquid or gel applied directly to a fingernail 104, such as via a brush or other applicator. If the fingernail 104 is currently covered or coated, it may be cleaned to provide a clean surface upon which the foundation layer 204 may be applied. In general, the foundation layer 204 provides a layer or substrate upon which the other layers or sections of the sparkling fingernail coating may be applied. In one or more embodiments, the foundation layer 204 may provide stronger adhesion to the fingernail 104 than other layers of the sparkling fingernail coating. Typically, the foundation layer 204 will be clear or transparent. This allows the fingernail 104 to be seen if desired. It is noted that the foundation layer 204 may be pigmented in some embodiments, such as to match the color of a higher layer of the sparkling fingernail coating.

At a step 308, the foundation layer 204 may be cured for a predefined period of time to solidify the foundation layer. In general, the period of time will be such that the foundation layer 204 is fully cured or set (e.g., fully dried and/or hardened) when curing is complete. Typically, it will take 30 seconds to 1 minute to fully cure the foundation layer 204.

Curing may be aided by various curing devices. For example, heat, moving air, and/or light may be applied to a client's fingernail 104 to cure the foundation layer 204 (or other layer). This is beneficial in that it can speed up the curing process. Also, in some embodiments, heat or light may activate certain compounds within the foundation layer 204 (or other layer) to allow for a desired or improved final product.

In one or more embodiments, a curing device may comprise a nail dryer. Such a curing device may include one or more heating elements, light emitters (such as UV or infrared light bulbs), or both to respectively apply heat and/or light to a client's fingernail 104. In addition or alternatively, a fan or the like may be provided to move air around the client's fingernail 104. The curing device may include an enclosure configured to accept one or more fingers 112 so as to allow one or multiple fingernails 104 to be cured at once. For instance, a client may cure all his or her fingernails 104 at once with this configuration.

It is noted that the predefined curing period may be adjusted based on the type of curing device used. For example, a UV light may only require 30 seconds to fully cure the foundation layer 204, while other light sources may need 1 minute to fully cure the foundation layer.

At a step 312, a glitter adhesion layer 208 may be applied to the foundation layer 204, such as shown in FIG. 2B. In one or more embodiments, the glitter adhesion layer 208 will be a distinct material composition as compared to the other layers of the sparkling fingernail coating. For instance, the foundation layer may be configured to secure or adhere the sparkling fingernail coating to a fingernail, while the glitter adhesion layer is configured to carry glitter particles embedded or suspended therein.

The glitter adhesion layer 208 may be applied in liquid or gel form. Typically, the glitter adhesion layer 208 will be applied (on top of the foundation layer) to cover the entire fingernail 104 including its tip. In one or more embodiments, the glitter adhesion layer 208 may be a thin layer relative to the foundation and other layers of the sparkling fingernail coating. In addition, in some embodiments, the glitter adhesion layer 208 may be tinted to various colors to impart a color to the sparkling fingernail coating.

At a step 316, the glitter adhesion layer 208 may be partially cured (e.g., heated or exposed to light or moving air) for a predefined period of time to prepare the glitter adhesion layer to accept and bond to glitter. This may occur by exposing the fingernail 204 to a curing device for the predefined period of time. Typically, heating will only occur for a short period of time to allows the glitter adhesion layer 208 to soften. For example, the glitter adhesion layer 208 may be heated for 5 to 10 seconds in some embodiments to prepare the glitter adhesion layer to accept and bond to glitter. The short period of time is not sufficient to fully cure the glitter adhesion layer thus preserving the glitter adhesion layer's ability to bond to glitter.

At a step 320, glitter 212 may be applied to the glitter adhesion layer 208. This may occur by sprinkling, spreading, or pouring individual glitter particles 212 onto the glitter adhesion layer. As shown in FIG. 2C for example, the glitter 212 is being applied across the glitter adhesion layer 208 with a brush 220. Typically, the glitter 212 will be applied to cover the entire glitter adhesion layer 208.

At a step 324, the glitter adhesion layer 208 (now carrying the glitter 212) may be cured for a predefined period of time to set the glitter. For example, the glitter adhesion layer 208 may be cured for 30 to 60 seconds to at least partially cure (e.g., dry and/or harden) the glitter adhesion layer.

At a step 328, the glitter 212 may be pressed into the glitter adhesion layer 208. For example, a brush 220 may be used to tap or press the glitter 212 into the glitter adhesion layer 208. The glitter 212 will typically be pressed in a direction perpendicular or normal to the surface of the fingernail 104. This presses the glitter 212 into the glitter adhesion layer 208, thus embedding the glitter without moving the glitter in a lateral direction. In cases where a brush is used, the nail technician may manipulate the brush handle 216 in such manner to press the glitter 212 as disclosed herein.

Once the glitter 212 has been pressed, the glitter adhesion layer 208 may be cured again for a predefined period of time at a step 332. Typically, this curing will fully cure (e.g., harden) the glitter adhesion layer 208. For example, the glitter adhesion layer 208 may be cured for another 30 to 60 seconds to fully cure the glitter adhesion layer 208.

With the sparkling fingernail coating, the glitter 212 is only manicured after the glitter adhesion layer 208 has been fully cured at step 332. Therefore, once fully cured, excess glitter 212 (as well as foundation layer or glitter adhesion layer material) may be removed. For example, excess glitter 212 may be removed from around the client's fingernail 104. This produces a sharp border for the sparkling fingernail coating that matches the peripheral shape of the client's fingernail 104. Removal of excess glitter 212 will typically only occur outside the client's fingernail 104. This prevents desired glitter 212 from becoming dislodged.

At a step 340, a top layer 224 may be applied to seal in the glitter 212 and prevent the glitter from becoming dislodged, such as shown in FIG. 2D. Typically the top layer 224 will be transparent so as to not impair visibility of the glitter 212. It is contemplated that the top layer 224 may be tinted various colors or be somewhat translucent if desired.

In general, the top layer 224 may be applied in a liquid or gel form to cover the entire fingernail 104, thus covering all the applied glitter 212. At a step 344, the top layer 224 may be cured so as to harden or dry the top layer. For example, the top layer 224 may be cured for 30 to 60 seconds to fully cure the top layer forming a completed sparkling fingernail coating. Once cured, the fingernail 104 may be filed to improve the shape of the sparkling fingernail coating 108. It is noted that filing may only occur after the top layer 224 has been applied and cured in one or more embodiments. This prevents the glitter 212 from becoming undesirably dislodged during filing.

At a step 348, the sparkling fingernail coating 108 may optionally be buffed to produce a smooth finish for the sparkling fingernail coating. It is noted that only the top layer 224 should be buffed. In other words, buffing should be discontinued if the top layer 224 has been buffed away at one or more locations of the fingernail 104.

At a decision step 352, if the sparkling fingernail coating 108 now has a smooth shiny or other desired finish, the application process may end at a step 356. If not, another top layer 224 may be applied at step 340, cured at step 344, and buffed at step 348 until a desired finish is achieved.

As can be seen, the use of filing and buffing is limited to later stages of the process to avoid undesirably dislodging any glitter 212. In addition, the various layers and curing stages give the sparkling fingernail coating 108 durability as well as a predictable lifetime before it becomes worn out or unsightly.

While various embodiments of the invention have been described, it will be apparent to those of ordinary skill in the art that many more embodiments and implementations are possible that are within the scope of this invention. In addition, the various features, elements, and embodiments described herein may be claimed or combined in any combination or arrangement.

1. A sparkling fingernail coating for a fingernail comprising:
a foundation layer applied to a top surface and tip of the fingernail;
a glitter adhesion layer applied to the foundation layer;
a plurality of glitter particles embedded in the glitter adhesion layer, wherein the glitter is embedded while the glitter adhesion layer is partially cured; and
a transparent top layer applied to the glitter adhesion layer, wherein the transparent top layer is applied to the glitter adhesion layer after the glitter adhesion layer is fully cured.

2. The sparkling fingernail coating of claim 1, wherein the foundation layer is transparent.

3. The sparkling fingernail coating of claim 2, wherein the glitter adhesion layer is tinted to a particular color.

4. The sparkling fingernail coating of claim 2, wherein the glitter adhesion layer is thinner than the foundation layer.

5. The sparkling fingernail coating of claim 2, wherein the glitter adhesion layer is transparent.

6. The sparkling fingernail coating of claim 1 further comprising at least one additional top layer.

7. The sparkling fingernail coating of claim 1, wherein at least the glitter adhesion layer is cured by curing device.

8. The sparkling fingernail coating of claim 1, wherein in the glitter adhesion layer is applied to the foundation layer after the foundation layer has fully cured.

9. A sparkling fingernail coating for a fingernail comprising:
a first layer configured to secure the sparkling fingernail coating to the fingernail;
a second layer configured to carry a plurality of glitter particles therein, wherein the second layer covers the first layer and is applied to the first layer after the first layer is fully cured;
a plurality of glitter particles embedded in the second layer, wherein the plurality of glitter particles are embedded after the second layer is partially cured;
one or more third layers covering the second layer and the plurality of glitter particles, wherein the one or more third layers are applied to cover the second layer and plurality of glitter particles after the second layer is fully cured.

10. The sparkling fingernail coating of claim 9, wherein the second layer is partially cured with UV light.

11. The sparkling fingernail coating of claim 9, wherein the second layer is fully cured with UV light.

12. The sparkling fingernail coating of claim 9, wherein the second layer is tinted to a particular color.

13. The sparkling fingernail coating of claim 12, wherein the one or more third layers are transparent.

14. The sparkling fingernail coating of claim 9, wherein the one or more third layers are buffed to a smooth finish.

15. A method for applying a sparkling fingernail coating to a fingernail comprising:
applying a foundation layer to a top surface and tip of the fingernail, the foundation layer configured to secure the sparkling fingernail coating to the fingernail;
covering the foundation layer with a glitter adhesion layer;
partially curing the glitter adhesion layer with a curing device for a first period of time to generate a partially cured glitter adhesion layer;
applying a plurality of glitter particles to the partially cured glitter adhesion layer;
embedding the plurality of glitter particles into the partially cured glitter adhesion layer;
fully curing the glitter adhesion layer with the curing device for a second period of time to generate a fully cured glitter adhesion layer having the plurality of glitter particles embedded therein; and
covering the fully cured glitter adhesion layer with a transparent top layer configured to protect the glitter adhesion layer.

16. The method of claim 15, wherein the second period of time is longer than the first period of time.

17. The method of claim 15 further comprising fully curing the foundation layer before the glitter adhesion layer is applied.

18. The method of claim 15 further comprising removing excess glitter outside a peripheral edge of the fingernail after the glitter adhesion layer has fully cured.

19. The method of claim 15 further comprising buffing the transparent top layer to generate a smooth finish.

20. The method of claim 15, wherein the curing device comprises a curing element selected from the group consisting of a UV light, heating element, and fan.
